SEQUENCE LISTING (4 sequences)

SEQ ID NO: 1 - DNA, 21 bp, synthetic construct
  source 1..21: mol_type = other DNA; note = forward primer for amplification of VDR
gtggacatcggcatgatgaag

SEQ ID NO: 2 - DNA, 21 bp, synthetic construct
  source 1..21: mol_type = other DNA; note = reverse primer for amplification of VDR
ggtcgtaggtcttatggtggg

SEQ ID NO: 3 - DNA, 18 bp, synthetic construct
  source 1..18: mol_type = other DNA; note = forward primer for amplification of GAPDH
gaaggtgaaggtcggagt

SEQ ID NO: 4 - DNA, 19 bp, synthetic construct
  source 1..19: mol_type = other DNA; note = reverse primer for amplification of GAPDH
gaagatggtgatggatttc

(19) United States
(12) Patent Application Publication
(10) Pub. No.: US 2024/0316127 A1
(43) Pub. Date: Sep. 26, 2024

(54) METHOD FOR INCREASING CALCIUM ABSORPTION USING CULTURE OF BIFIDOBACTERIUM ANIMALIS SUBSP. LACTIS CP-9

(71) Applicant: GLAC BIOTECH CO., LTD., Tainan City (TW)

(72) Inventors: Hsieh-Hsun HO, Tainan City (TW); Yi-Wei KUO, Tainan City (TW); Jui-Fen CHEN, Tainan City (TW); Chi-Huei LIN, Tainan City (TW); Shin-Yu TSAI, Tainan City (TW); Ko-Chiang HSIA, Tainan City (TW)

(21) Appl. No.: 18/513,826

(22) Filed: Nov. 20, 2023

(30) Foreign Application Priority Data
Mar. 24, 2023 (CN) 202310296585.5

(51) Int. Cl. A61K 35/745; A61K 33/06; A61K 35/747; A61P 3/14

(52) U.S. Cl. CPC A61K 35/745; A61K 33/06; A61K 35/747; A61P 3/14; C12R 2001/225

(57) ABSTRACT

A method for increasing calcium absorption includes administering to a subject in need thereof a composition containing a culture of Bifidobacterium animalis subsp. lactis CP-9. The Bifidobacterium animalis subsp. lactis CP-9 is deposited under the Budapest Treaty at the China Center for Type Culture Collection (CCTCC) under an accession number CCTCC M 2014588.

### CROSS-REFERENCE TO RELATED APPLICATION

This application claims priority to Chinese Invention Patent Application No. 202310296585.5, filed on Mar. 24, 2023, and incorporated by reference herein in its entirety.

### SEQUENCE LISTING XML

The Sequence Listing submitted concurrently herewith with a file name of “PE-68172-AM-SEQUENCE LISTING.xml,” a creation date of Jul. 26, 2023, and a size of 5.14 kilobytes, is part of the specification and is incorporated by reference in its entirety.

### FIELD

The present disclosure relates to a method for increasing calcium absorption using a composition including a culture of Bifidobacterium animalis subsp. lactis CP-9.

### BACKGROUND

Calcium deficiency-associated disorders are part of the global health issues and are common in children, women, and aged men. Calcium deficiency can diminish bone strength, potentially leading to rickets in children, osteomalacia in adults, osteoporosis, etc. The former two are more commonly caused by a lack of vitamin D while the latter is more frequently caused by a deficiency of calcium. A prevalent way to increase calcium intake for delaying or improving a calcium deficiency-associated disorder is through the use of calcium supplements, such as calcium carbonate, calcium oxide, calcium phosphate, and calcium lactate gluconate. Nevertheless, calcium contained in calcium supplements may not be absorbed effectively by the intestine due to a lack of vitamin D while excessive use of calcium supplements may increase urinary calcium levels, resulting in a high risk of kidney stones, which are made up of 80% to 90% calcium in the form of either calcium oxalate or calcium phosphate.

Probiotics are resident normal flora of the intestinal tract and are believed to play important roles in regulating proper intestinal immunity and digestion by balancing intestinal microflora. These beneficial microorganisms are widely used as live microbial dietary supplements and can help restoring intestinal microfloral balance. Many species of lactic acid bacteria (LAB), such as Lactobacillus spp., Lactococcus spp., Pediococcus spp., Streptococcus spp., Enterococcus spp., Bifidobacterium spp., Bacillus spp., and Leuconostoc spp., are conferred with the generally recognized as safe (GRAS) status, and are widely used as probiotics.

Previous studies have demonstrated that certain strains of LAB are capable of preventing osteoporosis. For example, CN 110964656 B discloses that Bifidobacterium animalis subsp. lactis BL-99, which is deposited under the Budapest Treaty at the China General Microbiological Culture Collection Center (CGMCC) under an accession number CGMCC 15650, can reduce the loss of bone mass caused by estrogen deficiency, and increase blood calcium ion and phosphorus ion concentration in ovariectomized rats, hence being capable of alleviating osteoporosis.

In spite of the aforesaid, there is still a need to develop an effective way for increasing calcium absorption.

### SUMMARY

Therefore, an object of the present disclosure is to provide a method for increasing calcium absorption, which can alleviate at least one of the drawbacks of the prior art, and which includes administering to a subject in need thereof a composition including a culture of Bifidobacterium animalis subsp. lactis CP-9.

The Bifidobacterium animalis subsp. lactis CP-9 is deposited under the Budapest Treaty at the China Center for Type Culture Collection (CCTCC) under an accession number CCTCC M 2014588.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the relative fold change in vitamin D receptor (VDR) gene expression determined in the Ca control group, experimental group, and comparative groups 1 to 3 of Example 1, infra, in which the symbol “*” represents p<0.05 (compared with the Ca control group).

FIG. 2 shows the relative fold change in calcium ion transport determined in the Ca control group, experimental group B, and comparative group B of Example 2, infra, in which the symbol “*” represents p<0.05 (compared with the Ca control group).

FIG. 3 shows the relative fold change in calcium ion transport determined in the Ca control group, experimental group S, and comparative group S of Example 2, infra, in which the symbol “**” represents p<0.01 (compared with the Ca control group).

FIG. 4 shows the relative fold change in calcium ion transport determined in the Ca control group, experimental group M, and comparative groups M1 and M2 of Example 3, infra, in which the symbol “**” represents p<0.05 (compared with the Ca control group).

FIG. 5 shows the relative fold change in calcium ion transport determined in the Ca control group and experimental groups 1 to 4 of Example 3, infra, in which the symbol “**” represents p<0.01 (compared with the Ca control group).

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for increasing calcium absorption, which includes administering to a subject in need thereof a composition including a culture of Bifidobacterium animalis subsp. lactis CP-9.

The Bifidobacterium animalis subsp. lactis CP-9 is deposited under the Budapest Treaty at the China Center for Type Culture Collection (CCTCC) under an accession number CCTCC M 2014588.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

In certain embodiments, the subject suffers from a calcium deficiency-associated disorder.

According to the present disclosure, the calcium deficiency-associated disorder may be selected from the group consisting of osteoporosis, osteopenia, rickets, joint pain, muscle cramp, hypertension, palpitation, anxiety, obesity, skin inflammation, hypocalcemia, dysmenorrhea, perimenopause, menopause, postmenopause, and combinations thereof.

According to the present disclosure, the culture of the lactic acid bacterial strain (i.e., the Bifidobacterium animalis subsp. lactis CP-9) may be prepared by culturing the abovementioned lactic acid bacterial strain in a liquid or solid medium suitable for growth and/or proliferation thereof.

As used herein, the term “culturing” can be used interchangeably with other terms such as “fermentation” and “cultivation”.

According to the present disclosure, the liquid medium suitable for cultivation may include, but is not limited to, MRS (De Man, Rogosa and Sharpe) broth, MRS broth containing cysteine, and mineral medium containing glucose and soy peptone.

The procedures and conditions for cultivation may be adjusted according to practical requirements. In this regard, those skilled in the art may refer to journal articles, e.g., Hsieh P. S. et al. (2013), New Microbiol., 36:167-179.

According to the present disclosure, cultivation may be conducted at a temperature ranging from 25° C. to 40° C. for a time period ranging from 20 hours to 40 hours. In an exemplary embodiment, cultivation is conducted at 37° C. for 24 hours.

According to the present disclosure, the culture of the lactic acid bacterial strain is a liquid culture.

According to the present disclosure, the liquid culture may have a total bacterial concentration ranging from 10⁶ CFU/mL to 10¹² CFU/mL. In certain embodiments, the liquid culture may have a total bacterial concentration ranging from 10⁶ CFU/mL to 10¹⁰ CFU/mL. In an exemplary embodiment, the liquid culture may have a total bacterial concentration of 9×10⁶ CFU/mL. In another exemplary embodiment, the liquid culture may have a total bacterial concentration of 9×10⁷ CFU/mL.

According to the present disclosure, the liquid culture may be free of cells.

As used herein, the term “free of” means that the liquid culture lacks a significant amount of a specified component (i.e., bacterial cells). In certain embodiments, the amount of the bacterial cells does not have a measurable effect on the properties of the liquid culture. In other embodiments, the liquid culture is completely free of the bacterial cells.

According to the present disclosure, the liquid culture which is free of cells is obtained by subjecting a culture formed after culturing the lactic acid bacterial strain to a solid-liquid separation.

According to the present disclosure, the solid-liquid separation treatment may be performed using techniques well-known to those skilled in the art. Examples of the solid-liquid separation treatment may include, but are not limited to, a centrifugation treatment, a filtration treatment, a concentration treatment, and combinations thereof. In an exemplary embodiment, the solid-liquid separation treatment is a centrifugation treatment.

According to the present disclosure, the liquid culture may be subjected to a sterilization treatment, and hence may be free of viable bacterial cells.

According to the present disclosure, the culture which contains bacterial cells only is obtained by subjecting a culture formed after culturing the lactic acid bacterial strain to the aforesaid solid-liquid separation treatment so as to remove a liquid portion therefrom.

According to the present disclosure, the culture may be concentrated or non-concentrated, a liquid, a paste, a semi-solid, or a solid (e.g., a pellet, a granule, or a powder), and may be heat-inactivated, frozen, dried, or freeze-dried (e.g., may be in freeze-dried form or spray/fluid bed dried form).

According to the present disclosure, the composition may further include a culture of a probiotic microorganism selected from the group consisting of Lactobacillus salivarius subsp. salicinius AP-32 (BCRC 910437; CCTCC M 2011127), Lactobacillus paracasei ET-66 (BCRC 910753; CGMCC 13514), Lactobacillus rhamnosus bv-77 (BCRC 910533; CCTCC M 2014589), and combinations thereof. In certain embodiments, the composition includes cultures of Bifidobacterium animalis subsp. lactis CP-9, Lactobacillus salivarius subsp. salicinius AP-32, and Lactobacillus paracasei ET-66.

In certain embodiments, the cultures of Bifidobacterium animalis subsp. lactis CP-9, Lactobacillus salivarius subsp. salicinius AP-32, and Lactobacillus paracasei ET-66 are liquid cultures which are free of cells. In addition, a volume ratio of the liquid cultures of Bifidobacterium animalis subsp. lactis CP-9, to Lactobacillus salivarius subsp. salicinius AP-32, and to Lactobacillus paracasei ET-66 in the composition ranges from 0.2:1:1 to 2:1:1. In an exemplary embodiment, the volume ratio of the liquid cultures of Bifidobacterium animalis subsp. lactis CP-9, to Lactobacillus salivarius subsp. salicinius AP-32, and to Lactobacillus paracasei ET-66 in the composition is 2:1:1.

According to the present disclosure, the composition may further include a calcium supplement. Examples of the calcium supplement may include, but are not limited to, calcium chloride, calcium carbonate, calcium oxide, calcium dihydrogen phosphate, dicalcium phosphate, tricalcium bis(phosphate), calcium lactate gluconate, calcium L-threonate, calcium citrate, calcium lactate, and combinations thereof. In an exemplary embodiment, the calcium supplement is calcium chloride.

According to the present disclosure, the composition may be formulated as a food product using a standard technique well known to one of ordinary skill in the art. For example, the composition may be directly added to an edible material or may be used to prepare an intermediate composition (e.g., a premix) suitable to be subsequently added to the edible material.

As used herein, the term “food product” refers to any article or substance that can be ingested by a subject into the body thereof. Examples of the food product may include, but are not limited to, milk powders, fermented milk, yogurt, butter, beverages (e.g., tea, coffee, etc.), functional beverages, a flour product, baked foods, confectionery, candies, fermented foods, animal feeds, health foods, infant foods, and dietary supplements.

According to the present disclosure, the composition may be prepared in the form of a pharmaceutical composition. The pharmaceutical composition may be formulated into a suitable dosage form for oral, parenteral or topical administration using technology well known to those skilled in the art.

According to the present disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

According to the present disclosure, the pharmaceutical composition may be formulated into an external preparation suitable for topical application to the skin using technology well known to those skilled in the art. The external preparation includes, but is not limited to, emulsions, gels, ointments, creams, patches, liniments, powders, aerosols, sprays, lotions, serums, pastes, foams, drops, suspensions, salves, and bandages.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intrapleural injection, intramuscular injection, intravenous injection, intraarterial injection, intraarticular injection, intrasynovial injection, intraepidermal injection, subcutaneous injection, intradermal injection, intralesional injection, and sublingual administration.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

According to the present disclosure, the dose and frequency of administration of the probiotic culture may vary depending on the following factors: the severity of the illness or disorder to be treated, routes of administration, and weight, age, physical condition and response of the subject to be treated. In general, the probiotic culture may be administered in a single dose or in several doses.

The disclosure will be further described by way of the following examples. However, it should be understood that the following examples are solely intended for the purpose of illustration and should not be construed as limiting the disclosure in practice.

### Examples

### General Experimental Materials:

### 1. Source and Cultivation of Human Colon Adenocarcinoma Cell Line Caco-2

Human colon adenocarcinoma cell line Caco-2 (ATCC® HTB-37™) was purchased from the American Type Culture Collection (ATCC, Manassas, Va., USA). The Caco-2 cells were grown in a 10-cm Petri dish containing Dulbecco's Modified Eagle's Medium (DMEM) (Cytiva) supplemented with 10% fetal bovine serum (FBS) and 1% penicillin-streptomycin (Cytiva). The Caco-2 cells were cultivated in an incubator at 37° C. with 5% CO₂. Medium change was performed every two to three days. Cell passage was performed when the cultured cells reached 80% to 90% of confluence.

### 2. Lactic Acid Bacterial (LAB) Strains

Bifidobacterium animalis subsp. lactis CP-9 (which is disclosed in the applicant's patent TW 1572713 B), Lactobacillus salivarius subsp. salicinius AP-32 (which is disclosed in the applicant's patent TW 1384990 B and CN 108338361 B) and Lactobacillus paracasei ET-66 (which is disclosed in the applicant's patent TW 1639389 B, TW 1812128 B, CN 108338361 B) are known and readily available to the public, and have been deposited at the Bioresource Collection and Research Center (BCRC) of the Food Industry Research and Development Institute (FIRDI) (No. 331, Shih-Pin Rd., Hsinchu City 300, Taiwan). In addition, Bifidobacterium animalis subsp. lactis CP-9 and Lactobacillus salivarius subsp. salicinius AP-32 have also been deposited under the terms of the Budapest Treaty on the International Recognition of the Deposit of Microorganisms for the Purpose of Patent Procedure at the International Depositary Authority, i.e., the China Center for Type Culture Collection (CCTCC) of Wuhan University, the College of Life Sciences (No. 299, Bayi Rd., Wuchang District, Wuhan City 430072, Hubei Province, China) in accordance with the Budapest Treaty, and Lactobacillus paracasei ET-66 has also been deposited under the terms of the Budapest Treaty on the International Recognition of the Deposit of Microorganisms for the Purpose of Patent Procedure at the International Depositary Authority, i.e. the China General Microbiological Culture Collection Center (CGMCC) of Chinese Academy of Sciences, the Institute of Microbiology (No. 1, West Beichen Rd., Chaoyang District, Beijing 100101, China) in accordance with the Budapest Treaty.

The relevant information regarding each of the three LAB strains (including accession number and date of deposit) is listed in Table 1 below.

TABLE 1

| LAB strains | Accession number | Date of deposit |
|---|---|---|
| Bifidobacterium animalis | BCRC 910645 | Aug. 21, 2014 |
| subsp. lactis CP-9 | CCTCC M 2014588 | Nov. 24, 2014 |
| Lactobacillus salivarius | BCRC 910437 | Jul. 30, 2009 |
| subsp. salicinius AP-32 | CCTCC M 2011127 | Apr. 10, 2011 |
| Lactobacillus paracasei | BCRC 910753 | Nov. 3, 2016 |
| ET-66 | CGMCC 13514 | Dec. 29, 2016 |

In addition, the following Bifidobacterium animalis subsp. lactis strains were used as comparative strains, including Bifidobacterium animalis subsp. lactis BL-99 purchased from Yili Industrial Group Co., Ltd, Bifidobacterium animalis subsp. lactis BB-12 purchased from Chr. Hansen A/S, Denmark, and Bifidobacterium animalis subsp. lactis gL-53 isolated by the applicant from breast milk of a healthy human.

### 3. Preparation of Bacterial Suspension and Cell Culture Supernatant of LAB Strains Containing 5 mM Calcium Chloride

A respective one of the six LAB strains described in section 2 of “General Experimental Materials” was inoculated in a MRS broth (Difco, Cat. no. 288130) supplemented with 0.05% cysteine (Sigma-Aldrich, Cat. no. C7352), followed by cultivation in an incubator (37° C., 5% CO₂) under an anaerobic condition for 24 hours to obtain a respective inoculum. Thereafter, the respective inoculum was inoculated in an amount of 2% (v/v) into a MRS broth supplemented with 0.05% cysteine, followed by cultivation in an incubator (37° C., 5% CO₂) under an anaerobic condition for 24 hours. After centrifugation at 3,000 rpm and 4° C. for 10 minutes, the resultant cell culture supernatant and cell pellet were collected. The cell culture supernatant was then added an appropriate amount of calcium chloride (Sigma-Aldrich, Cat. no. V900269), so as to obtain a cell culture supernatant containing 5 mM calcium chloride (abbreviated as CaCl₂-cell culture supernatant). With regard to the cell pellet, an appropriate amount of DMEM supplemented with 5 mM calcium chloride was added to suspend the cell pellet and adjust to the desired bacterial concentration which was determined using a plate counting medium, thereby obtaining a bacterial suspension containing 5 mM calcium chloride and a bacterial concentration of 9×10⁷ CFU/mL (abbreviated as CaCl₂-bacterial suspension). The resultant CaCl₂-cell culture supernatants and CaCl₂-bacterial suspensions of the aforesaid LAB strains were used in the following experiments.

### General Procedures:

### 1. Statistical Analysis

All the experiments described below were performed in triplicates.

The experimental data of all the test groups are expressed as mean±standard deviation (SD), and were analyzed using one-way analysis of variance (ANOVA), so as to evaluate the differences between the groups. Statistical significance is indicated by p<0.05.

### Example 1. Evaluation of the Effect of Bifidobacterium animalis Subsp. Lactis CP-9 on Increasing Expression of Vitamin D Receptor (VDR) in Intestinal Cells

In this example, the expression level of vitamin D receptor (VDR) was determined, so as to evaluate the effect of Bifidobacterium animalis subsp. lactis CP-9 on improving calcium absorption.

### Experimental Procedures:

First, the Caco-2 cells prepared in section 1 of “General Experimental Materials” were divided into 6 groups, including a blank control group, a Ca control group, an experimental group, and three comparative groups (i.e., comparative groups 1 to 3). Each group of the Caco-2 cells was seeded at a concentration of 2×10⁵ cells/well into a respective well of 6-well culture plates containing 3 mL of DMEM supplemented with 10% FBS and 1% penicillin-streptomycin, followed by cultivation in an incubator (37° C., 5% CO₂) for 11 days. During the cultivation period, the culture medium in each group was removed to be replaced with a fresh culture medium every three days. At the end of the cultivation period, the culture medium in each well was removed. The cells of each of the experimental group and the comparative groups were added with 3 mL of the respective one of the CaCl₂-bacterial suspensions prepared in section 3 of “General Experimental Materials” as shown in Table 2 below. In addition, the cells of each of the blank control group and the Ca control group were added with 3 ml of the respective testing agent as shown in Table 2 below, followed by cultivation in an incubator (37° C., 5% CO₂) for 6 hours.

TABLE 2

| Group | Testing agent |
|---|---|
| Blank control group | DMEM |
| Ca control group | DMEM containing 5 mM CaCl₂ |
| Experimental group | CaCl₂-bacterial suspension of Bifidobacterium |
| | animalis subsp. lactis CP-9 |
| Comparative group 1 | CaCl₂-bacterial suspension of Bifidobacterium |
| | animalis subsp. lactis BL-99 |
| Comparative group 2 | CaCl₂-bacterial suspension of Bifidobacterium |
| | animalis subsp. lactis BB-12 |
| Comparative group 3 | CaCl₂-bacterial suspension of Bifidobacterium |
| | animalis subsp. lactis gL-53 |

Thereafter, the liquid in each well was removed and the cell culture of each group was subjected to total RNA extraction using a Total RNA Extraction Miniprep System (Viogene, Cat. no. GR1001) in accordance with the manufacturer's instructions. The resultant RNA of each group was used as a template for synthesizing cDNA by reverse transcription polymerase chain reaction (RT-PCR) using GoScript™ Reverse Transcriptase (Promega, Cat. no. A5003) in accordance with the manufacturer's instructions.

The thus obtained cDNA, serving as a DNA template, was subjected to quantitative real-time PCR based on SYBR-Green I fluorescence, which was performed on a StepOnePlus™ real-time PCR system (Applied Biosystems™) using a designed primer pair specific for VDR gene shown in Table 3 and the reaction conditions shown in Table 4. Glyceraldehyde-3-phosphate dehydrogenase (GAPDH) gene was used as an endogenous control in the quantitative analysis of real-time PCR to normalize the gene expression data.

TABLE 3

| | | | Size of |
|---|---|---|---|
| | | Nucleotide | PCR |
| | PCR | sequence | product |
| Target gene | primer | (5′→3′) | (bp) |
| VDR gene | Forward | gtggacatcggcat | 181 |
| (GenBank | primer | gatgaag | |
| accession no. | VDR-F | (SEQ ID NO: 1) | |
| NM_000376.3) | Reverse | ggtcgtaggtctta | |
| | primer | tggtggg | |
| | VDR-R | (SEQ ID NO: 2) | |
| GAPDH gene | Forward | gaaggtgaaggtcg | 225 |
| (NCBI | primer | gagt | |
| accession no. | GAPDH-F | (SEQ ID NO: 3) | |
| NM_002046.2) | Reverse | gaagatggtgatgg | |
| | primer | atttc | |
| | GAPDH-R | (SEQ ID NO: 4) | |

TABLE 4

| | Volume |
|---|---|
| Reaction mix | (μL) |
| cDNA (0.1 μg/μL) | 2 |
| Forward primer (10 μM) | 0.8 |
| Reverse primer (10 μM) | 0.8 |
| qPCRBIO SyGreen Mix (PCR Biosystems, Cat. no. | 10 |
| PB20.12-05) | |
| Deionized water | 6.4 |

Operating conditions: forty cycles of the following reactions: denaturation at 95° C. for 3 minutes, annealing at 95° C. for 5 seconds, and extension at 60° C. for 30 seconds.

The resultant PCR product was subjected to determination of fluorescence intensity, followed by calculating the cycle threshold (Cₜ) value of VDR gene. The Cₜ value of VDR gene in each group was normalized with that of GAPDH gene using the comparative Cₜ method, and the relative fold change in VDR gene expression for each group was further calculated using the following Equation (1):

$$A = ( B - C ) / ( D - C ) \qquad ( 1 )$$

where A=relative fold change in VDR gene expression
B=normalized Cₜ value of VDR gene in each group
C=normalized Cₜ value of VDR gene in the blank control group
D=normalized Cₜ value of VDR gene in the Ca control group

The data thus obtained were analyzed according to the procedures as described in section 1 of “General Procedures”.

### Results:

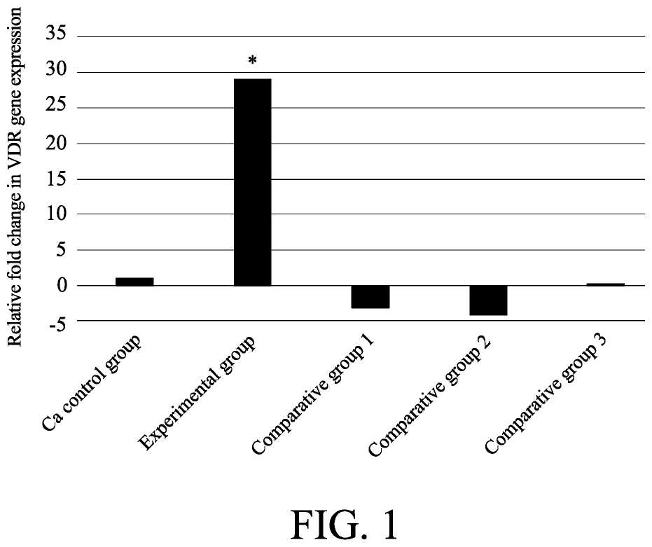

FIG. 1 shows the relative fold change in VDR gene expression determined in each group after treating the Caco-2 cells with the respective testing agent as shown in Table 2. As shown in FIG. 1, compared with the Ca control group, the relative fold change in VDR gene expression determined in the experimental group showed a significant increase, and that of the comparative group 3 had no significant change, whereas those of the comparative group 1 and the comparative group 2 showed significant decreases. These results demonstrate that Bifidobacterium animalis subsp. lactis CP-9 can effectively increase the VDR expression in intestinal cells while other Bifidobacterium animalis subsp. lactis strains not only fail to increase the VDR expression, but also may inhibit the VDR expression.

### Example 2. Evaluation of the Effect of Bifidobacterium animalis Subsp. Lactis CP-9 on Improving Intestinal Calcium Ion Transport

Based on the results obtained in Example 1 above, the applicant selected Bifidobacterium animalis subsp. lactis gL-53 to be compared with Bifidobacterium animalis subsp. lactis CP-9 in terms of improvement in intestinal calcium ion transport.

### Experimental Procedures:

First, the Caco-2 cells prepared in section 1 of “General Experimental Materials” were divided into 6 groups, including a blank control group, a Ca control group, two experimental groups (i.e., experimental group B and experimental group S), and two comparative groups (i.e., comparative group B and comparative group S). Each group of the Caco-2 cells was seeded at a concentration of 2×10⁵ cells per well into a respective permeable Transwell® inserts (Corning Inc.) for 6-well plates. Each of the Transwell® inserts had a polycarbonate membrane (pore size: 0.4 μm) and contained 2.5 mL of DMEM. Next, the Transwell® inserts were placed into the 6-well plates containing 1.5 ml of DMEM in each well, followed by cultivation in an incubator (37° C., 5% CO₂) for 6 days, and medium change was performed every 3 days, so that the Caco-2 cells formed a cell monolayer in the Transwell® inserts. Afterwards, the culture medium in each Transwell® insert was removed, and the respective Transwell® insert was washed with phosphate-buffered saline (PBS) two times. The Caco-2 cell monolayer of each of the experimental groups B, the experimental group S, the comparative group B, and the comparative group S was added with 1.5 mL of the respective one of the CaCl₂-bacterial suspensions or CaCl₂-cell culture supernatants prepared in section 3 of “General Experimental Materials” as shown in Table 5 below. In addition, the Caco-2 cell monolayer of each of the blank control group and the Ca control group was added with 1.5 mL of the respective testing agent as shown in Table 5 below.

TABLE 5

| Group | Testing agent |
|---|---|
| Blank control group | DMEM |
| Ca control group | DMEM containing 5 mM CaCl₂ |
| Experimental | CaCl₂-bacterial suspension of Bifidobacterium |
| group B | animalis subsp. lactis CP-9 |
| Experimental | CaCl₂-cell culture supernatant of Bifidobacterium |
| group S | animalis subsp. lactis CP-9 |
| Comparative | CaCl₂-bacterial suspension of Bifidobacterium |
| group B | animalis subsp. lactis gL-53 |
| Comparative | CaCl₂-cell culture supernatant of Bifidobacterium |
| group S | animalis subsp. lactis gL-53 |

Each group was cultivated in an incubator (37° C., 5% CO₂) for 24 hours. On the 30th minute and 24th hour after the treatment with the CaCl₂-bacterial suspension or CaCl₂-cell culture supernatant, the liquid in each well was collected, and was then subjected to determination of calcium content using a Calcium Colorimetric Assay Kit (BioVision, Cat. No. K380-250).

The percentage increase in calcium content for each group was calculated by substituting the detected calcium content on the 30th minute and 24th hour after the treatment into the following Equation (2):

$$E = F / G \qquad ( 2 )$$

where E=percentage increase in calcium content
F=calcium content detected in each group on the 24th hour after the treatment
G=calcium content detected in each group on the 30th minute after the treatment

The relative fold change in calcium ion transport for each group was calculated by substituting the thus obtained percentage increase in calcium content into the following Equation (3):

$$H = ( I - J ) / ( K - J ) \qquad ( 3 )$$

where H=relative fold change in calcium ion transport
I=percentage increase in calcium content for each group
J=percentage increase in calcium content for the blank control group
K=percentage increase in calcium content for the Ca control group

The data thus obtained were analyzed according to the procedures as described in section 1 of “General Procedures”.

### Results:

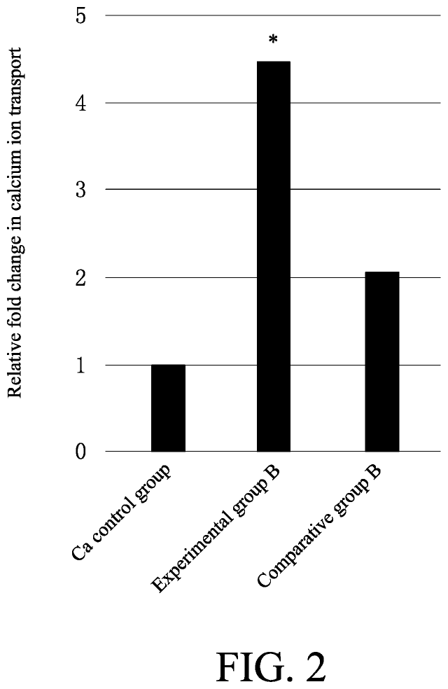

FIG. 2 shows the relative fold change in calcium ion transport determined in each of the Ca control group, experimental group B, and comparative group B after treating the Caco-2 cell monolayer with the respective testing agent as shown in Table 5. As shown in FIG. 2, compared with the Ca control group, the relative fold change in calcium ion transport determined in the comparative group B only showed a slight increase without statistical significance, whereas the relative fold change in calcium ion transport determined in the experimental group B showed a significant increase. In addition, the relative fold change in calcium ion transport determined in the experimental group B was more than twice as high as that of the comparative group B.

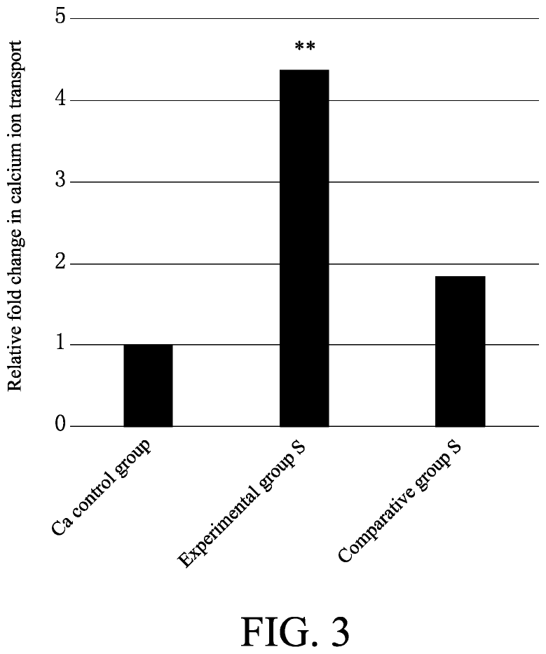

FIG. 3 shows the relative fold change in calcium ion transport determined in each of the Ca control group, experimental group S, and comparative group S after treating the Caco-2 cell monolayer with the respective testing agent as shown in Table 5. As shown in FIG. 3, the relative fold change in calcium ion transport of the experimental group S was also more than twice compared with that of the comparative group S. These results demonstrate that both the CaCl₂-bacterial suspension and the CaCl₂-cell culture supernatant of Bifidobacterium animalis subsp. lactis CP-9 can effectively improve intestinal calcium ion transport and the effects are significantly better than other Bifidobacterium animalis subsp. lactis strains.

Summarizing the above test results, it is clear that compared with other Bifidobacterium animalis subsp. lactis strains, the culture of Bifidobacterium animalis subsp. lactis CP-9 (including the bacterial cells portion and the cell culture supernatant without bacterial cells) significantly increases the expression of the VDR and calcium ion transport in intestinal cells, and hence can effectively increase calcium absorption.

### Example 3. Evaluation of the Effect of Supernatant Mixture of LAB Strain on Improving Intestinal Calcium Ion Transport

In order to evaluate the effect of Bifidobacterium animalis subsp. lactis CP-9 in combination with other probiotic lactic acid bacterial strains on improving intestinal calcium ion transport, the following experiments were conducted.

A. Effect of a Combination of Bifidobacterium animalis Subsp. Lactis CP-9 and Other Probiotics on Intestinal Calcium Ion Transport

First, the CaCl₂-cell culture supernatants of Bifidobacterium animalis subsp. lactis CP-9, Lactobacillus salivarius subsp. salicinius AP-32, Lactobacillus paracasei ET-66, and Bifidobacterium animalis subsp. lactis BL-99 prepared in section 3 of “General Experimental Materials” were mixed in specific combinations and volume ratios as shown in Table 6 below, so as to obtain 3 supernatant mixtures (i.e., supernatant mixtures 1 to 3).

TABLE 6

| Supernatant | | Volume |
|---|---|---|
| mixture | LAB strain applied | ratio |
| 1 | Bifidobacterium animalis subsp. lactis CP-9, | 1:1:1 |
| | Lactobacillus salivarius subsp. salicinius | |
| | AP-32, and Lactobacillus paracasei ET-66 | |
| 2 | Lactobacillus salivarius subsp. salicinius AP-32 | 1:1 |
| | and Lactobacillus paracasei ET-66 | |
| 3 | Bifidobacterium animalis subsp. lactis BL-99, | 1:1:1 |
| | Lactobacillus salivarius subsp. salicinius AP- | |
| | 32, and Lactobacillus paracasei ET-66 | |

The Caco-2 cells prepared in section 1 of “General Experimental Materials” were divided into 5 groups, including a blank control group, a Ca control group, an experimental group (i.e., experimental group M), and two comparative groups (i.e., comparative groups M1 and M2). Each group of the Caco-2 cells was seeded at a concentration of 2×10⁵ cells per well into a respective permeable Transwell® inserts (Corning Inc.) for 6-well plates. Each of the Transwell® inserts had a polycarbonate membrane (pore size: 0.4 μm) and contained 2.5 mL of DMEM. Next, the Transwell® inserts were placed into the 6-well plates containing 1.5 mL of DMEM in each well, followed by cultivation in an incubator (37° C., 5% CO₂) for 6 days, and medium change was performed every 3 days, so that the Caco-2 cells formed a cell monolayer in the Transwell® inserts. Afterwards, the culture medium in each Transwell® insert was removed, the respective Transwell® insert was washed with phosphate-buffered saline (PBS) two times. Thereafter, the Caco-2 cell monolayer of each of the experimental group M and the comparative groups M1 and M2 was added with 1.5 mL of the respective supernatant mixture as shown in Table 7 below. In addition, the Caco-2 cell monolayer of each of the blank control group and the Ca control group was added with 1.5 ml of the respective testing agent as shown in Table 7 below.

TABLE 7

| Group | Testing agent |
|---|---|
| Blank control group | DMEM |
| Ca control group | DMEM containing 5 mM CaCl₂ |
| Experimental group M | Supernatant mixture 1 |
| Comparative group M1 | Supernatant mixture 2 |
| Comparative group M2 | Supernatant mixture 3 |

Each group was cultivated in an incubator (37° C., 5% CO₂) for 24 hours. On the 30th minute and 24th hour after the treatment with the testing agent, the liquid in each well was collected, and was then subjected to determination of calcium content and relative fold change in calcium ion transport according to the procedures described in “Experimental Procedures” of Example 2.

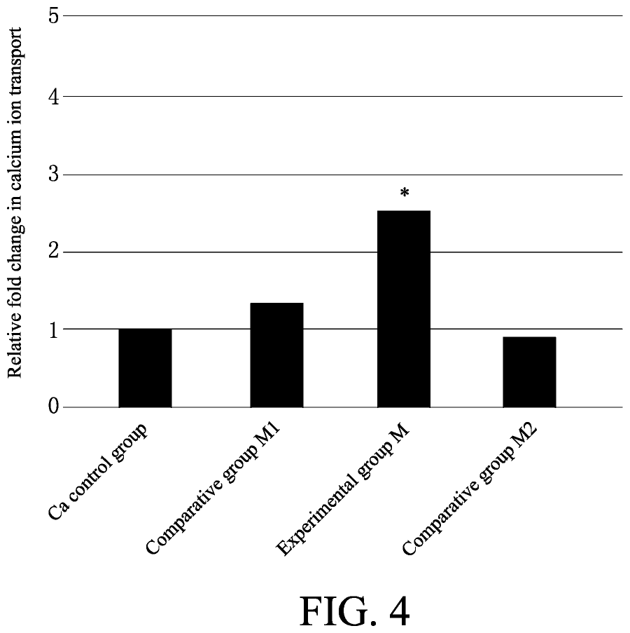

FIG. 4 shows the relative fold change in calcium ion transport determined in each of the Ca control group, experimental group M, and comparative groups M1 and M2 after treating the Caco-2 cell monolayer with the respective testing agent as shown in Table 7. As shown in FIG. 4, compared with the Ca control group, the relative fold change in calcium ion transport determined in both the comparative groups M1 and M2 showed no significant change, whereas that of the experimental group M showed a significant increase. These results demonstrate that Bifidobacterium animalis subsp. lactis CP-9 of the present disclosure is expected to be useful for improving intestinal calcium ion transport, and may be further used in combination with other known probiotics to exert desirable effects.

B. Effect of Different Volume Ratios of Bifidobacterium animalis Subsp. Lactis CP-9 in Combination with Other Probiotics on Intestinal Calcium Ion Transport

First, the CaCl₂-cell culture supernatants of Bifidobacterium animalis subsp. lactis CP-9, Lactobacillus salivarius subsp. salicinius AP-32, and Lactobacillus paracasei ET-66 prepared in section 3 of “General Experimental Materials” were mixed in different volume ratios as shown in Table 8 below, so as to obtain 4 supernatant mixtures (i.e., supernatant mixtures 1 to 4).

TABLE 8

| Supernatant | | Volume |
|---|---|---|
| mixture | LAB strain applied | ratio |
| 1 | Bifidobacterium animalis subsp. lactis CP-9, | 0:1:1 |
| | Lactobacillus salivarius subsp. salicinius | |
| | AP-32, and Lactobacillus paracasei ET-66 | |
| 2 | Bifidobacterium animalis subsp. lactis CP-9, | 0.2:1:1 |
| | Lactobacillus salivarius subsp. salicinius | |
| | AP-32, and Lactobacillus paracasei ET-66 | |
| 3 | Bifidobacterium animalis subsp. lactis CP-9, | 0.85:1:1 |
| | Lactobacillus salivarius subsp. salicinius | |
| | AP-32, and Lactobacillus paracasei ET-66 | |
| 4 | Bifidobacterium animalis subsp. lactis CP-9, | 2:1:1 |
| | Lactobacillus salivarius subsp. salicinius | |
| | AP-32, and Lactobacillus paracasei ET-66 | |

The Caco-2 cells prepared in section 1 of “General Experimental Materials” were divided into 6 groups, including a blank control group, a Ca control group, and four experimental groups (i.e., experimental groups 1 to 4). Each group of the Caco-2 cells was seeded at a concentration of 2×10⁵ cells per well into a respective permeable Transwell® inserts (Corning Inc.) for 6-well plates. Each of the Transwell® inserts had a polycarbonate membrane (pore size: 0.4 μm) and contained 2.5 mL of DMEM. Next, the Transwell® inserts were placed into the 6-well plates containing 1.5 mL of DMEM in each well, followed by cultivation in an incubator (37° C., 5% CO₂) for 6 days, and medium change was performed every 3 days, so that the Caco-2 cells formed a cell monolayer in the Transwell® inserts. Afterwards, the culture medium in each Transwell® insert was removed, the respective Transwell® insert was washed with phosphate-buffered saline (PBS) two times. Thereafter, the Caco-2 cell monolayer of each of the experimental groups 1 to 4 was added with 1.5 mL of the respective supernatant mixture as shown in Table 9 below. In addition, the Caco-2 cell monolayer of each of the blank control group and the Ca control group was added with 1.5 mL of the respective testing agent as shown in Table 9 below.

TABLE 9

| Group | Testing agent |
|---|---|
| Blank control group | DMEM |
| Ca control group | DMEM containing 5 mM CaCl₂ |
| Experimental group 1 | Supernatant mixture 1 |
| Experimental group 2 | Supernatant mixture 2 |
| Experimental group 3 | Supernatant mixture 3 |
| Experimental group 4 | Supernatant mixture 4 |

Each group was cultivated in an incubator (37° C., 5% CO₂) for 24 hours. On the 30th minute and 24th hour after the treatment with the testing agent, the liquid in each well was collected, and was then subjected to determination of calcium content and relative fold change in calcium ion transport according to the procedures described in “Experimental Procedures” of Example 2.

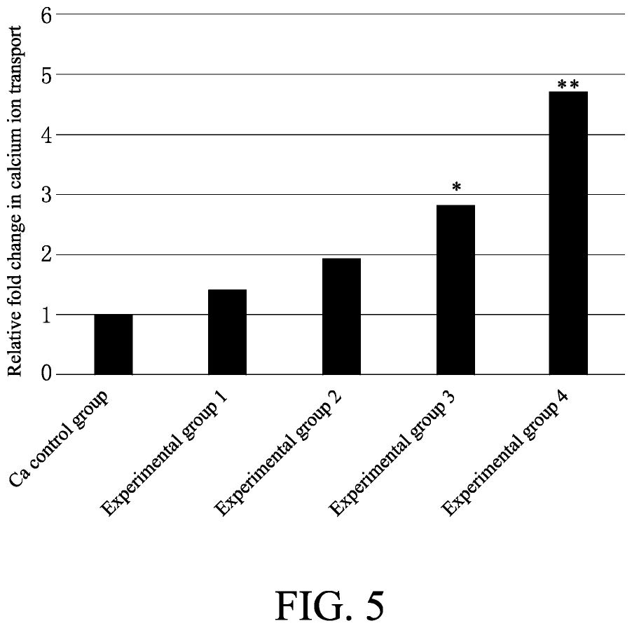

FIG. 5 shows the relative fold change in calcium ion transport determined in each of the Ca control group and experimental groups 1 to 4 after treating the Caco-2 cell monolayer with the respective testing agent as shown in Table 9. As shown in FIG. 5, compared with the Ca control group, the relative fold changes in calcium ion transport determined in the experimental groups 1 to 4 tended to notably increase with increasing proportions of Bifidobacterium animalis subsp. lactis CP-9 in the supernatant mixtures (i.e., the supernatant mixtures 1 to 4). In particular, compared with the Ca control group, the relative fold change in calcium ion transport determined in the experimental group 4 showed a significant increase. In addition, the relative fold change in calcium ion transport determined in the experimental group 4 was more than three times as high as that of the experimental group 1. These results indicate that Bifidobacterium animalis subsp. lactis CP-9, Lactobacillus salivarius subsp. salicinius AP-32, and Lactobacillus paracasei ET-66, when mixed in a specified volume ratio ranging from 0.2:1:1 to 2:1:1, can exhibit satisfactory efficacy in improving intestinal calcium ion transport.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for increasing calcium absorption, comprising administering to a subject in need thereof a composition including a culture of Bifidobacterium animalis subsp. lactis CP-9,
wherein the Bifidobacterium animalis subsp. lactis CP-9 is deposited under the Budapest Treaty at the China Center for Type Culture Collection (CCTCC) under an accession number CCTCC M 2014588.

2. The method as claimed in claim 1, wherein the culture is a liquid culture.

3. The method as claimed in claim 2, wherein the liquid culture is free of cells.

4. The method as claimed in claim 1, wherein the culture contains bacterial cells only.

5. The method as claimed in claim 1, wherein the subject suffers from a calcium deficiency-associated disorder.

6. The method as claimed in claim 1, wherein the composition further includes a calcium supplement.

7. The method as claimed in claim 1, wherein the composition is a food product or a pharmaceutical composition.

8. The method as claimed in claim 7, wherein the pharmaceutical composition is administered by a route selected from the group consisting of oral administration, parenteral administration, and topical administration.

9. The method as claimed in claim 1, wherein the composition further includes a culture of a probiotic microorganism selected from the group consisting of Lactobacillus salivarius subsp. salicinius AP-32 which is deposited under the Budapest Treaty at the CCTCC under an accession number CCTCC M 2011127, Lactobacillus rhamnosus bv-77 which is deposited under the Budapest Treaty at the CCTCC under an accession number CCTCC M 2014589, and Lactobacillus paracasei ET-66 which is deposited under the Budapest Treaty at the China General Microbiological Culture Collection Center (CGMCC) under an accession number CGMCC 13514, and combinations thereof.

10. The method as claimed in claim 9, wherein the composition includes cultures of Bifidobacterium animalis subsp. lactis CP-9, Lactobacillus salivarius subsp. salicinius AP-32, and Lactobacillus paracasei ET-66.

11. The method as claimed in claim 10, wherein the cultures of Bifidobacterium animalis subsp. lactis CP-9, Lactobacillus salivarius subsp. salicinius AP-32, and Lactobacillus paracasei ET-66 are liquid cultures which are free of cells.

12. The method as claimed in claim 11, wherein a volume ratio of the liquid cultures of Bifidobacterium animalis subsp. lactis CP-9, to Lactobacillus salivarius subsp. salicinius AP-32, and to Lactobacillus paracasei ET-66 in the composition ranges from 0.2:1:1 to 2:1:1.
